(19) United States
(12) Patent Application Publication
(10) Pub. No.: US 2026/0199049 A1
(43) Pub. Date: Jul. 16, 2026

(54) POSITION GAUGE FOR SURGICAL INSTRUMENTS

(71) Applicant: Alliance Veterinary Surgery, PLLC, Glen Cove, NY (US)

(72) Inventor: Jeffrey A. SEAMAN, Glen Cove, NY (US)

(73) Assignee: Alliance Veterinary Surgery, PLLC, Glen Cove, NY (US)

(21) Appl. No.: 19/023,786

(22) Filed: Jan. 16, 2025

(51) Int. Cl. A61B 90/00; A61B 17/14; A61B 17/16

(52) U.S. Cl. CPC A61B 90/06; A61B 17/14; A61B 17/16; A61B 2090/068

(57) ABSTRACT

A position gauge for a surgical instrument is formed from a base plate having an upper surface and a lower surface, a pin extending from the lower surface of the base plate at an angle perpendicular to a plane of the lower surface of the base plate; and a level arranged on the upper surface of the base plate. The level is a circular level having an enclosed chamber filled with liquid and a gas bubble disposed in the chamber. A position of the gas bubble in a center of the chamber indicates a position perpendicular to horizontal. The chamber contains a marking that indicates the position perpendicular to horizontal, when the bubble is located within the marking. The position gauge is inserted into a channel in a motorized surgical instrument so that the level can indicate the correct vertical position of the instrument to the surgeon during surgery.

### BACKGROUND OF THE INVENTION

### 1. Field of the Invention

This invention relates to a position gauge for surgical instruments. In particular, the invention relates to a device that can indicate whether the instrument is properly positioned patient prior to use, so that the surgeon can make adjustments prior to cutting.

### 2. The Prior Art

Hand-held motorized surgical instruments, such as drills and saws, require precise placement in the patient in order to perform the necessary surgery without undue trauma or complications. It is often important that the handpiece be positioned so that the surgical instrument is positioned at exactly 90 degrees or at another desired angle to the working surface. However, since the instrument is hand-held, it is often difficult for the surgeon to achieve that precise angle using just their vision and judgment.

### SUMMARY OF THE INVENTION

It is therefore an object of the invention to provide a way for the surgeon to determine the precise angular position of the surgical instrument prior to and during operation of the surgical instrument. It is another object to provide such a device that is simple and inexpensive to manufacture.

These and other objects are accomplished by a position gauge for a surgical instrument that is formed from a base plate having an upper surface and a lower surface, a pin extending from the lower surface of the base plate at an angle perpendicular to a plane of the lower surface of the base plate; and a level arranged on the upper surface of the base plate. The level is preferably a circular level comprising an enclosed chamber filled with liquid and a gas bubble disposed in the chamber, wherein a position of the gas bubble in the center of the chamber indicates the level position, which also then indicates the vertical position of the pin, which is mounted perpendicular to horizontal. Preferably, the chamber is substantially cylindrical. However, it is possible to achieve proper positioning with chambers of other shapes. The position of the bubble at the center of the of the chamber is what indicates the proper position of the instrument. Preferably, the chamber contains a marking that indicates the level position when the bubble is located within the marking. This marking can be a circle, or any other type of marking that shows the perpendicular position of the level. The markings can also be calibrated for specific angles other than 90 degrees.

The positioning gauge can be made of any suitable material. The pin is preferably made of metal, and the chamber of the level is preferably of transparent plastic. The materials chosen should be chosen for durability as well as for easy of cleaning and/or sterilizing.

The positioning gauge is configured for use with most commercially available hand-held motorized surgical tools, such as the Synthes Unium^{TM}. The invention thus also relates a surgical instrument in which the positioning gauge is installed. The surgical instrument comprises a housing having a main body with a front end and a rear end and a channel extending longitudinally through the main body. A motor is disposed in the housing, and a hand grip extends from the main body. The housing also contains a power supply such as a battery or a connection to an electrical or pneumatic power system. A surgical tool is positionable at the front end of the main body. This tool can be a drill bit, a saw, a burr, or any other type of surgical tool attachment that is used with a motor-driven tools. The pin of the position gauge is disposed in the channel at the rear end of the main body, so that when the tool is aimed so that the tool attachment is facing the patient, the level is facing the surgeon. It is often required that the tool be positioned exactly vertical when making certain cuts or drill holes. The pin is placed in the channel so that the level sits on top of the tools in the user position. The level offers a precise physical measurement of the angle of the surgical tool at all times during use. At any time if the bubble deviates from the indicated marking, the surgeon can see exactly how to reposition the tool, by moving the tool until the bubble in the level is again within the indicated marking.

The position gauge can also be used with other types of surgical instruments that may not have a vertical channel for the mounting pin of the invention. To accommodate these other tools, a second embodiment of the invention includes a mounting element for receiving the position gauge. The mounting element has holes along one lateral side for accommodating a user’s fingers. The user places their fingers through the holes and then grips the instrument being used, which is intended to be placed in an exactly vertical position. The mounting element can alternatively be attached to the tool by straps or other suitable means. The position gauge is mounted in a channel of the mounting element, that extends longitudinally along the side opposite the finger holes, so that the level sits on the top surface of the mounting element, facing the user, and lets the user see if the instrument moves away from the vertical position during use.

The position gauge according to the invention is made to be easily placed and removed in the surgical tool, so that it can be used with other surgical tools, and so that the surgical tool can accommodate long tool attachments that may extend entirely through the channel and beyond the rear of the surgical tool.

### BRIEF DESCRIPTION OF THE DRAWINGS

Other objects and features of the present invention will become apparent from the following detailed description considered in connection with the accompanying drawings. It is to be understood, however, that the drawings are designed as an illustration only and not as a definition of the limits of the invention.

In the drawings, wherein similar reference characters denote similar elements throughout the several views:

FIG. 1 shows a perspective view of the position gauge according to the invention;

FIG. 2 shows a side view of the position gauge;

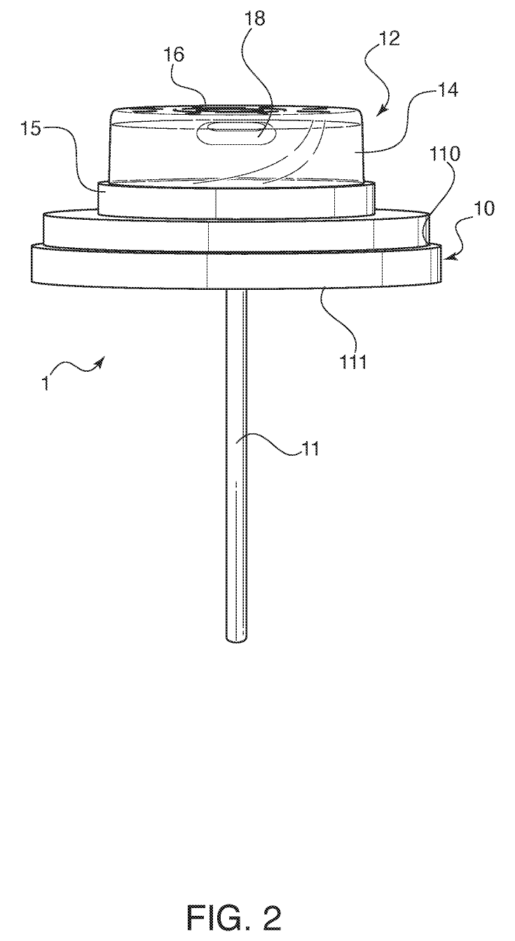

FIG. 3 shows the positioning gauge mounted on a rotary drive;

FIG. 4 shows another view of the position gauge mounted on the rotary drive;

FIG. 5 shows a mounting element for use with the position gauge according to the invention;

FIG. 6 shows a side view of the mounting element; and

FIG. 7 shows the mounting element with the position gauge inserted therein, and in use with a medical instrument.

### DETAILED DESCRIPTION OF THE EMBODIMENTS

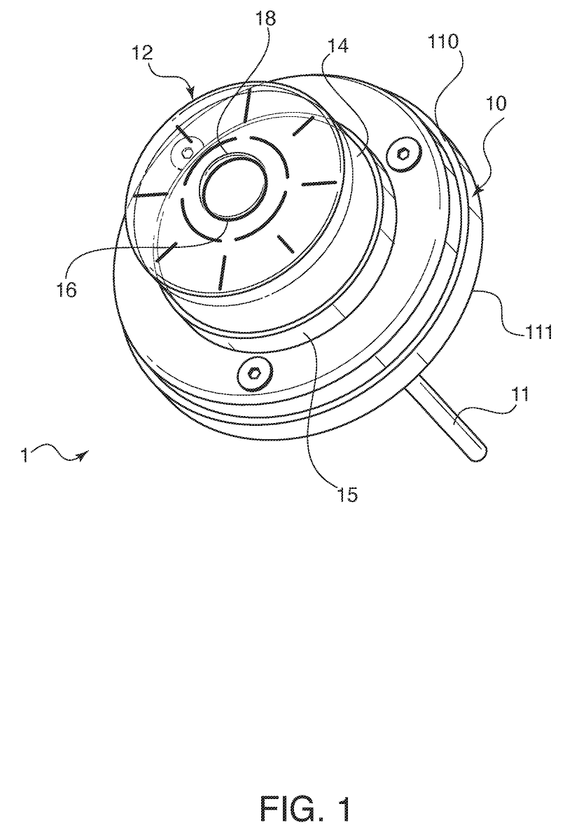

Referring now in detail to the drawings, FIG. 1 shows the position gauge 1 according to the invention, which has a base plate 10 having an upper surface 110 and a lower surface 111, a pin 11 extending from lower surface 111 of base plate 10 at an angle perpendicular to a plane of lower surface 111 of the base plate 10, and a level 12 arranged on upper surface 110 of the base plate 10. Level 12 is a circular level comprising an enclosed cylindrical chamber 14 on a bottom 15. Bottom 15 can be formed of several layers or a single layer and is connectable to base plate 10 via an suitable means, such as by screws or adhesive. Chamber 14 is filled with liquid and has a gas bubble 18 disposed in chamber 14. The position of gas bubble 18 in the center of chamber 14 indicates a position where level 12 is exactly horizontal, and as such the pin 11 is perpendicular to horizontal. Chamber 14 has markings 16 to indicate the proper position of gas bubble 18. Markings 16 can be a circle or any other shape or combination of shapes that would indicate a center of the level. In this embodiment, the position of bubble 19 within the inner circle indicates that the positioning gauge is level.

Pin 11 has a length that is at least as long as the diameter of base plate 10, to allow for secure positioning during use. The pin has a diameter that is suitable for insertion into the channel of most hand-held surgical instruments. In some cases, the pin has a diameter of 1/8 in., but other diameters could be used for channels of varying sizes.

Chamber 14 is substantially cylindrical, but could be of any desired shape. The position of bubble 18 at the center of chamber 14 as indicated by markings 16 is what indicates the proper position of the instrument, i.e., wherein pin 11 is aimed directly vertically at the patient.

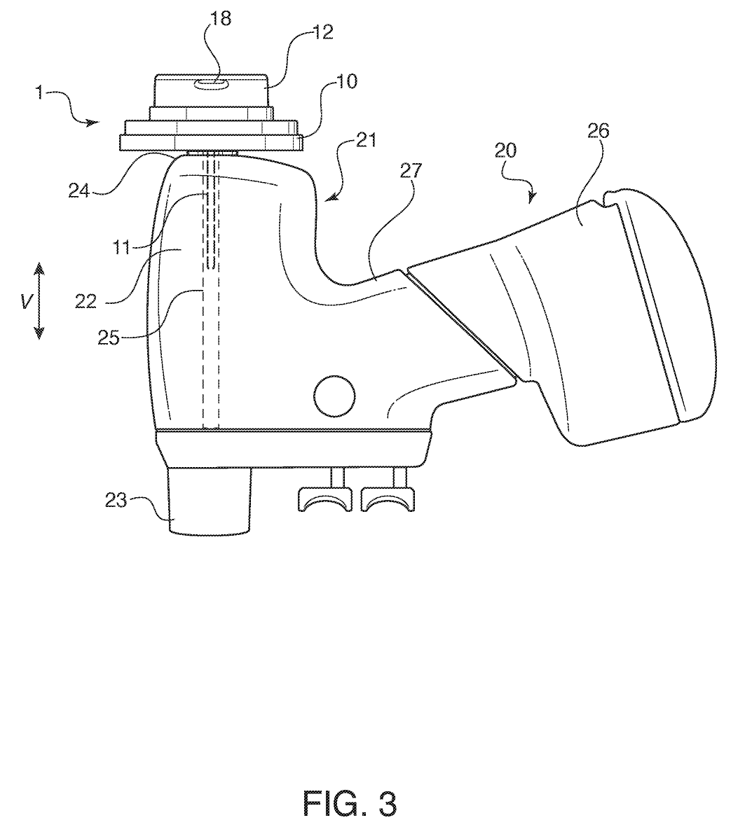

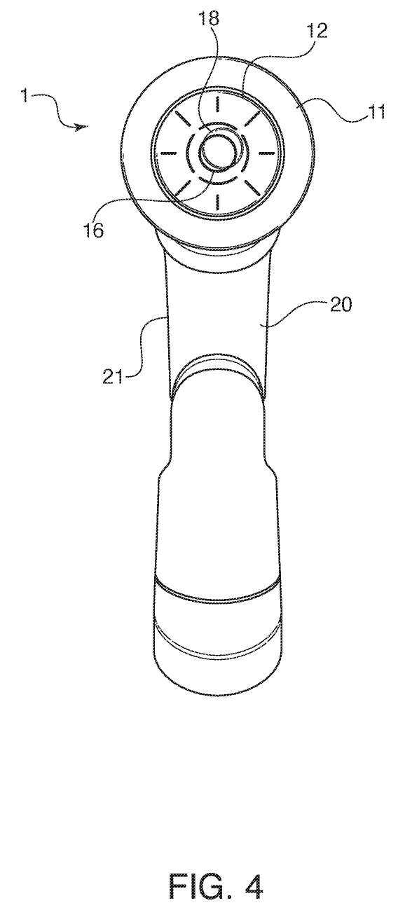

FIG. 3 shows position gauge 1 in used on a surgical instrument 20. Surgical instrument 20 comprises a housing 21 having a main body 22 with a front end 23 and a rear end 24 and a channel 25 extending longitudinally through main body 22. A motor is disposed in the housing, and a hand grip 27 extends from main body 22. Housing 21 also contains a power supply 26 such as a battery. A surgical tool (not shown) is positionable in front end 23 of main body 22. The tool can be a drill bit, a saw, a burr, or any other type of surgical tool attachment that is used with motor-driven instruments. Pin 11 of position gauge 1 is disposed in channel 25 at the rear end 24 of main body 22, so that when tool 20 is aimed so that the attachment is facing the patient, level 12 is facing the surgeon. It is often required that tool 20 be positioned exactly vertically, i.e., perpendicular to horizontal, when making certain cuts or drill holes. Pin 11 is placed in channel 25 so that the level sits on top of the tool 20 in the user position, as shown in FIG. 4. Pin 11 is arranged perpendicular to the plane of base plate 10.

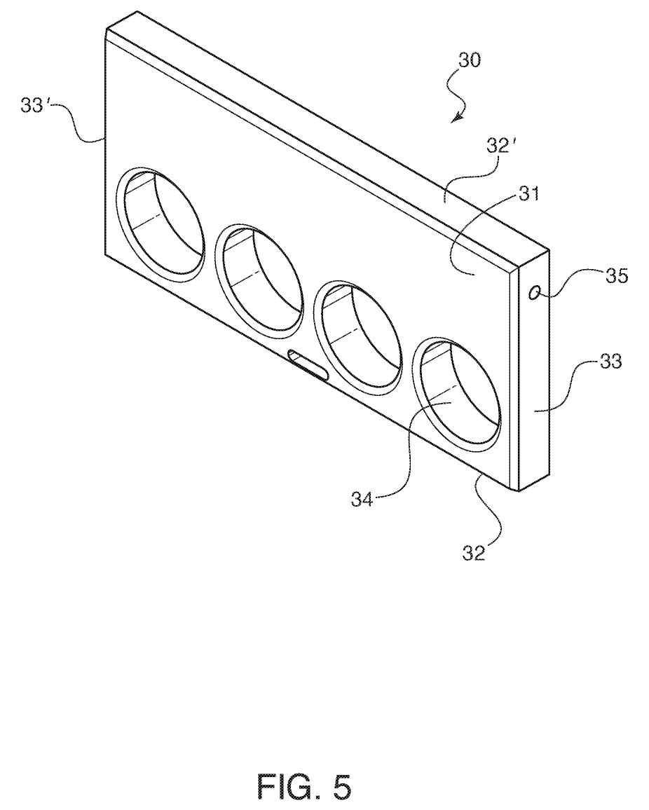

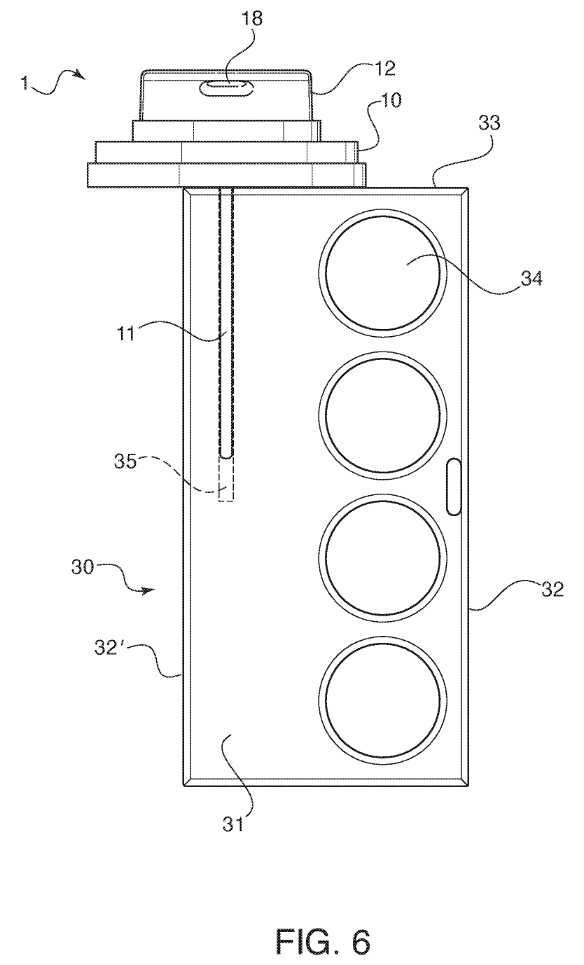

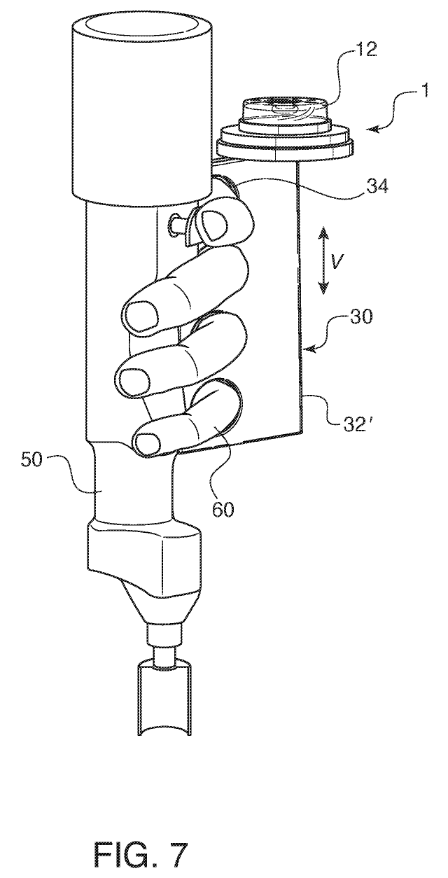

In situations where the surgical instrument being used does not have a channel for placement of the position gauge 1, a mounting element 30 can be used to hold position gauge 1 during use. Mounting element 30 is shown in FIGS. 5-7. Mounting element 30 has front and back flat surfaces 31, longitudinal sides 32, 32’ and end faces 33., 33’. Four finger holes 34 extend through flat surfaces 31 adjacent one longitudinal side 32, and a channel 35 extends longitudinally adjacent longitudinal side 32’ along mounting element 30, opening into end face 33. As shown in FIGS. 6 and 7, position gauge 1 can be placed on mounting element 30 by placing pin 11 into channel 35, so that position gauge 1 rests on top of end face 33.

Position gauge 1 in use with mounting element 30 is shown in FIG. 7. For example, for use with a saw 50, position gauge 1 is placed in channel 35 of mounting element 30. The user places their fingers 60 through finger holes 34 in mounting element 30, and then grips saw 60 in the position for normal operation. During use, level 12 is facing the surgeon and indicates if the saw 50 becomes tilted in any way, as the desired position is exactly vertical, as indicated by arrow V. In this position, bubble 18 is located within marking 16 (shown in FIG. 1). Any time bubble 18 moves outside the central circle of marking 16, the surgeon will know that the saw 50 is out of alignment and must be moved back until the bubble 18 is back within the center of marking 16.

Position gauge 1 can be easily inserted and removed from surgical instrument 20 or from mounting element 30, so that it is transferable between multiple instruments.

Accordingly, while only a few embodiments of the present invention have been shown and described, it is obvious that many changes and modifications may be made thereunto without departing from the spirit and scope of the invention.

What is claimed is:

1. A position gauge for a surgical instrument comprising:
a base plate having an upper surface and a lower surface;
a pin extending from the lower surface of the base plate at an angle perpendicular to a plane of the lower surface of the base plate; and
a level arranged on the upper surface of the base plate.

2. The position gauge according to claim 1, wherein the level is a circular level comprising an enclosed chamber filled with liquid and a gas bubble disposed in the chamber, wherein a position of the gas bubble in a center of the chamber indicates a position perpendicular to horizontal.

3. The position gauge according to claim 2, wherein the chamber is substantially cylindrical.

4. The position gauge according to claim 2, wherein the chamber contains a marking that indicates the position perpendicular to horizontal when the bubble is located within the marking.

5. The position gauge according to claim 1, wherein the pin is dimensioned to fit in an opening at a rear of the surgical instrument.

6. The position gauge according to claim 1, wherein the pin has a length that is at least as great as a diameter of the base plate.

7. The position gauge according to claim 1, further comprising a mounting element configured for attachment to the position gauge, the mounting element having a front surface, a rear surface, two end faces and two lateral sides, and a plurality of holes extending through the mounting base from the front surface to the rear surface, the holes being configured to accommodate a user’s fingers therein,
the mounting base further comprising a channel extending into the mounting base from one of the end faces and being configured to accommodate the mounting pin of the position gauge, such that when a user places their fingers through the holes and grips an instrument, the level faces upward and indicates a position of the instrument relative to vertical.

8. The position gauge according to claim 7, wherein the plurality of holes comprises four holes.

9. The position gauge according to claim 7, wherein the holes are arranged adjacent one of the longitudinal sides.

10. The position gauge according to claim 7, wherein the channel is located adjacent the longitudinal side that is opposite the side adjacent the holes.

11. The position gauge according to claim 7, wherein the position gauge is removable from the channel.

12. A surgical instrument comprising:
a housing having a main body with a front end and a rear end, and a channel extending longitudinally through the main body,
a motor disposed in the housing;
a hand grip extending from the main body;
a surgical tool attached to the front end of the main body, and
the position gauge according to claim 1, wherein the pin of the position gauge is disposed in the channel at the rear end of the main body.

13. The surgical instrument according to claim 12, wherein the position gauge is not fixed in the channel and is removable.
